(19) United States
(12) Patent Application Publication
(10) Pub. No.: US 2015/0182109 A1
(43) Pub. Date: Jul. 2, 2015

(54) SUBJECTIVE OPTOMETER

(71) Applicant: KABUSHIKI KAISHA TOPCON, Itabashi-ku,Tokyo (JP)

(72) Inventor: Tomohiro Sakurada, Itabashi-ku (JP)

(73) Assignee: KABUSHIKI KAISHA TOPCON, Itabashi-ku,Tokyo (JP)

(21) Appl. No.: 14/415,850

(22) PCT Filed: Aug. 9, 2013
(86) PCT No.: PCT/JP2013/071631

§ 371 Date: Jan. 20, 2015

(30) Foreign Application Priority Data
Aug. 31, 2012 (JP) 2012-192193

(51) Int. Cl. A61B 3/028

(52) U.S. Cl. CPC A61B 3/028

(57) ABSTRACT

A subjective optometer includes a visual target, deflectors, calculator and positioning parts. The visual target is presented a first distance away from eyes. The deflectors deflect first optical paths formed between the eyes and the visual target to form second optical paths causing the eyes to observe the visual target as if the visual target is presented at a second distance shorter than the first distance. The calculator calculates accommodation stimulus amount for causing the eyes to observe the visual target as if presented at the second distance based on convergence accommodation amount of the eyes caused by the second optical paths, the first distance and the second distance. The positioning parts include spherical lenses and position spherical lenses having spherical powers corresponding to the accommodation stimulus amount in the second optical paths. The eyes are examined with the spherical lenses positioned in the second optical paths.

### TECHNICAL FIELD

The present invention relates to a subjective optometer.

### BACKGROUND TECHNOLOGY

A subjective optometer is an apparatus that presents visual targets (eye charts) to an eye and examines visual functions of the eye based on responses regarding visual performance from a subject.

Examinations using subjective optometers include two types of examinations, namely, long range examinations for examining distant vision and short range examinations for examining near vision. The long range examinations are examinations in which long range visual targets that are visual targets located at long range examination distance (in general, 5 meters or more away from an eye). The short long range examinations are examinations in which short range visual targets that are visual targets located at short range examination distance (40 centimeters away from an eye, for example). Here, the examination distances are distances from a line connecting both eyes to visual targets.

Therefore, it is necessary to prepare two kinds of visual targets, namely long range visual targets and short range visual targets, for carrying out these two kinds of examinations hitherto. Further, subjective optomaters capable of carrying out long range examinations and short range examinations using one kind of visual targets have existed.

It is necessary for the subjective optomaters capable of carrying out long range examinations and short range examinations using one kind of visual targets to apply convergence stimuli and accommodation stimuli in accordance with an examination distance to the eyes. The subjective optomaters realizes a state in which the eyes observe the examination distance by such an action. Thus, this apparatus causes convergence of optic axes of the eyes corresponding to a state in which the eyes observe short range examination distance by means of prism lenses, for example. Further, when human eyes observe a visual target located at predetermined examination distance, accommodation in accordance with the distance is caused. Therefore, this apparatus causes accommodation of eyes corresponding to the state in which short range examination distance are observed by means of spherical lenses, for example. In this manner, this apparatus carries out long range examinations and short range examinations in conditions in which convergence and accommodation of the eyes are caused corresponding to the states in which the examination distances are observed.

### PRIOR ART DOCUMENT

### Patent Document

[Patent Document 1] Japanese Laid-open Patent Publication No. 2008-148930

### SUMMARY OF THE INVENTION

### Problem to be Solved by the Invention

However, accommodation is induced by convergence of optic axes of eyes. This is a phenomenon called convergence accommodation. When applying both convergence stimuli and accommodation stimuli to eyes by means of conventional subjective optomaters, convergence accommodation of the eyes is caused by causing convergence of optic axes of the eyes at first, and then accommodation of the eyes in accordance with an examination distance is caused. In other words, in a case in which both convergence stimuli and accommodation stimuli described above are applied to eyes, conventional subjective optomaters cause excessive accommodation of eyes due to influence of convergence accommodation.

A purpose of the present invention is to provide subjective optomaters capable of reducing influence of convergence accommodation and carrying out examinations in a state in which convergence and accommodation of eyes are caused in accordance with a predetermined examination distance.

### Means for Solving the Problem

The invention of Claim 1 is a subjective optometer comprising: a visual target that is presented at a location a first distance away from eyes; deflectors that deflect first optical paths formed between the eyes and the visual target to form second optical paths for causing the eyes to observe the visual target as if the visual target is presented at a location a second distance away, wherein the second distance is shorter than the first distance; a calculator that calculates accommodation stimulus amount for causing the eyes to observe the visual target as if the visual target is presented at the second distance based on convergence accommodation amount of the eyes caused by the second optical paths, the first distance and the second distance; and positioning parts that comprise spherical lenses and position spherical lenses having spherical powers corresponding to the accommodation stimulus amount calculated by the calculator in the second optical paths, wherein the eyes are examined in a state in which the spherical lenses are positioned in the second optical paths.

The invention of Claim 2 is the subjective optometer of Claim 1, wherein the deflectors comprise prism lenses and the prism lenses refract the first optical paths to deflect the first optical paths.

The invention of Claim 3 is the subjective optometer of Claim 1, wherein the deflectors comprise: visual target optical systems in which an optical system forming optical distance from the visual target to an eye and the visual target are integrally configured for each of a left eye and a right eye; and a driver that drives the visual target optical systems, wherein the driver drives the visual target optical systems to deflect the first optical paths.

The invention of Claim 4 is the subjective optometer of any of Claims 1 to 3, wherein the calculator comprises a storage that previously stores accommodation power information indicating accommodation powers of the eyes, and calculates the convergence accommodation amount of the eyes caused by the second optical paths based on the accommodation powers indicated by the accommodation power information and the second distance.

The invention of Claim 5 is the subjective optometer of Claim 4, wherein the storage previously stores a range in the accommodation powers of the eyes occupied by the convergence accommodation amount of the eyes, and the calculator calculates the convergence accommodation amount of the eyes caused by the second optical paths based on an upper limit of the range stored in the storage, the accommodation powers indicated by the accommodation power information and the second distance.

### Effect of the Invention

A subjective optomater according to the present invention is capable of reducing influence of convergence accommodation and carrying out examinations in a state in which convergence and accommodation of eyes are caused in accordance with a predetermined examination distance.

### BRIEF EXPLANATION OF THE DRAWINGS

FIG. 1 is a block diagram illustrating an example of a configuration of a subjective optomater according to an embodiment.

FIG. 2 is a schematic diagram illustrating an outline of a subjective optomater according to an embodiment.

FIG. 3 is a schematic diagram illustrating an outline of a subjective optomater according to an embodiment.

FIG. 4 is a flowchart illustrating an example of operation of a subjective optomater according to an embodiment.

FIG. 5 is a block diagram illustrating an example of a configuration of a subjective optomater according to an embodiment.

FIG. 6 is a schematic diagram illustrating an outline of a subjective optomater according to an embodiment.

FIG. 7 is a schematic diagram illustrating an outline of a subjective optomater according to an embodiment.

### MODES FOR CARRYING OUT THE INVENTION

Examples of embodiments of subjective optomaters according to the present invention are explained with reference to drawings.

### First Embodiment

### Configuration

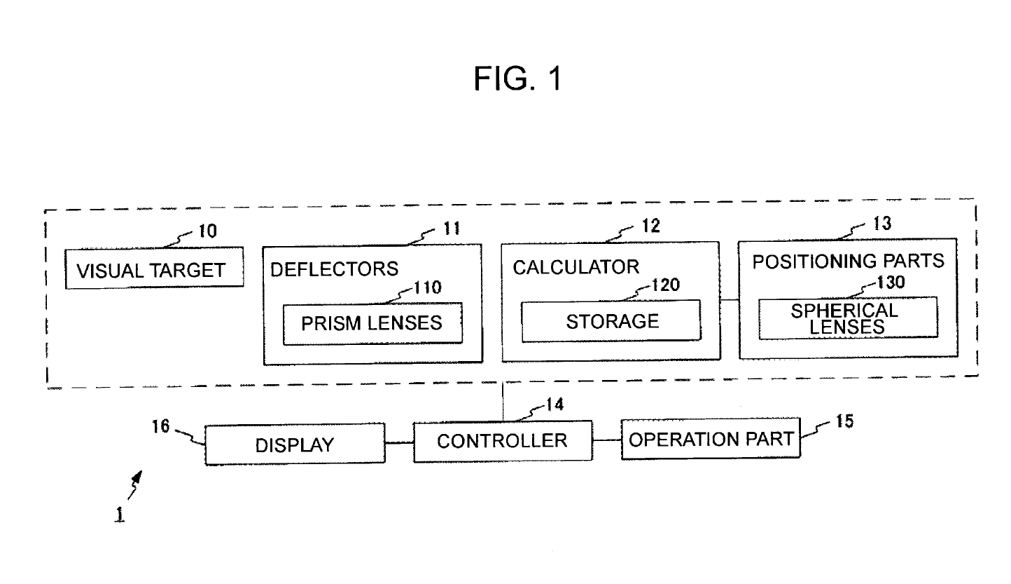

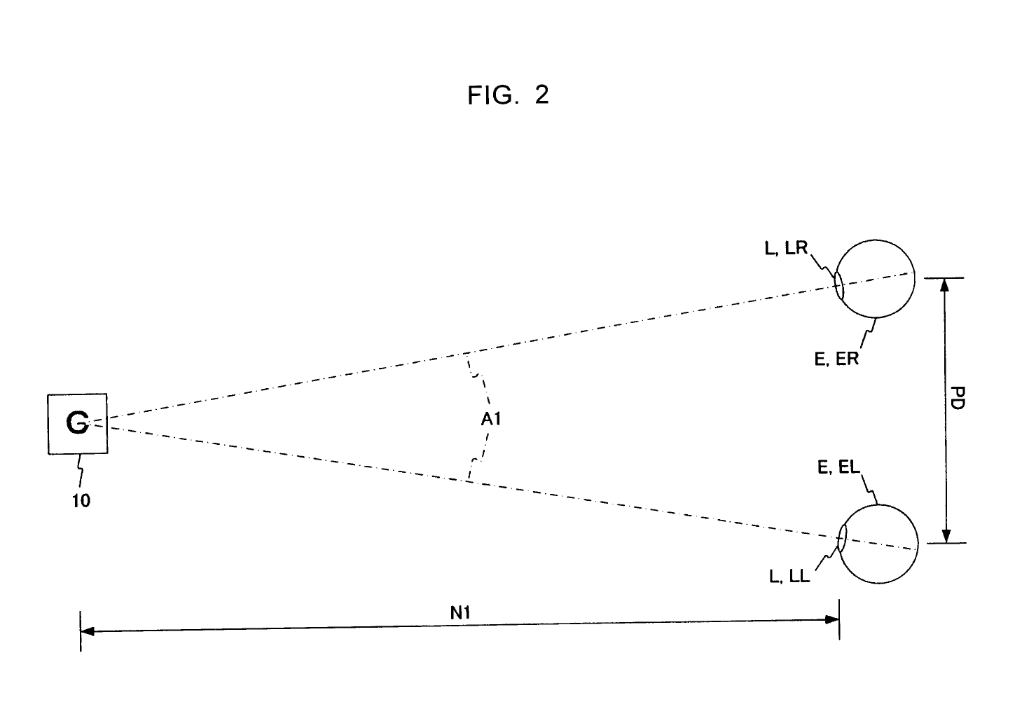

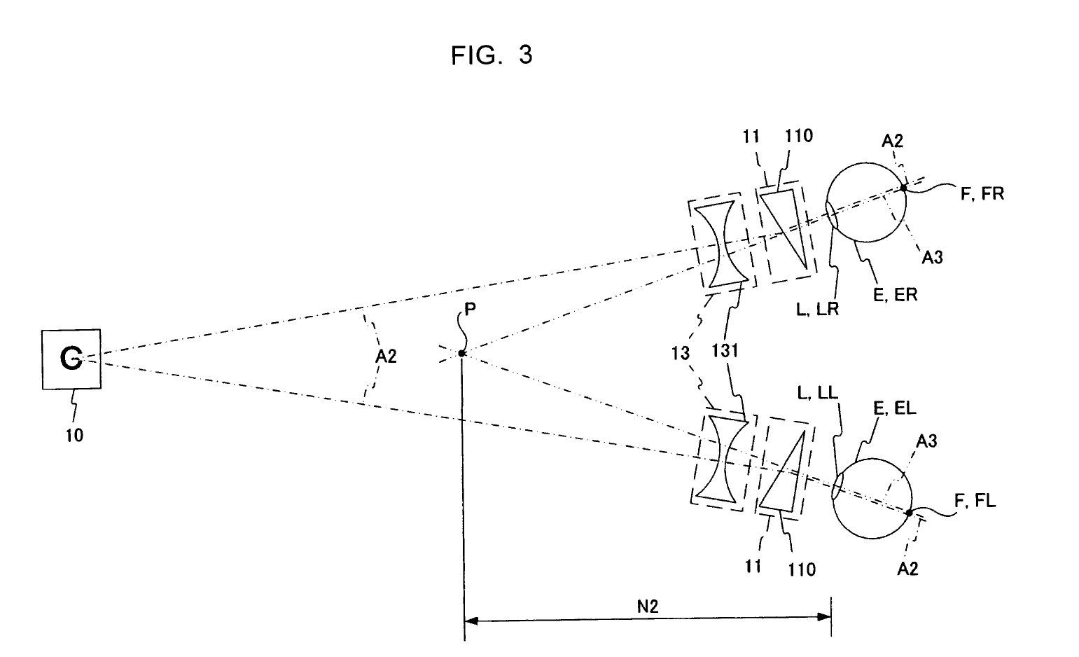

FIG. 1 is a block diagram illustrating a configuration of a subjective optometer 1 according to the present embodiment. FIG. 2 is a schematic diagram illustrating a state in which eyes E (a left eye EL and a right eye ER) observe a visual target 10 through first optical paths A1. FIG. 3 is a schematic diagram illustrating a state in which the eyes E observe the visual target 10 through second optical paths A2.

The subjective optometer 1 includes the visual target 10, deflectors 11, a calculator 12, positioning parts 13, a controller 14, an operation part 15 and a display 16.

### (Visual Target 10)

The visual target 10 is presented at a location that is first distance N1 away from the eyes E. Here, the first distance N1 is distance from a line connecting the both eyes E to the visual target 10. A subject observes the visual target 10 through the first optical paths A1 and answers a way how is is seen. The visual target 10 is presented to the eyes by a display device such as liquid crystal display, for example. Alternatively, the visual target 10 may be presented to the eyes by information printed on a paper.

### (Deflectors 11)

The deflectors 11 deflect the first optical paths A1 formed between the eyes E and the visual target 10 to form the second optical paths A2 for causing the eyes E to visually recognize the visual target 10 as if the visual target 10 is presented at second distance N2 that is shorter than the first distance N1. When the eyes E observe the visual target 10 through the second optical paths A2 formed by such deflection, optic axes A3 of the eyes E are converged so as to accord with the second optical paths A2. Further, since distance between an intersection P of the optic axes A3 of the eyes E and the line connecting the both eyes corresponds to the second distance N2, the eyes E observe the visual target 10 as if it is presented at the second distance N2. The optic axis A3 is an axis passing through a center of a crystalline lens L (a crystalline lens LL of the left eye or a crystalline lens LR of the right eye) and a central fovea F (a central fovea FL of the left eye or a central fovea FR of the right eye). It should be noted that the second distance N2 may be designated by an operator via the operation part 15 and the controller 14 described below.

Further, the deflectors 11 includes prism lenses 110 and the prism lenses 110 refract the first optical paths A1 to deflect the first optical paths A1. Prism amount (prism power) a of the prism lenses 110 is derived by means of the following equation.

$$α = ( \frac{\frac{PD}{2}}{N 2} - \frac{\frac{PD}{2}}{N 2} ) \times 100 \qquad [ Equation 1 ]$$

α: prism amount [Δ: prism diopter]
PD: distance between centers of rotations of eyes of a subject [mm: millimeters]
N1: first distance [mm]
N2: second distance [mm]

The deflectors 11 receives information indicating the distance PD between the centers of rotations of the eyes of the subject, the first distance N1 and the second distance N2 from the controller 14 described below and derives the prism amount α. the distance PD between the centers of rotations of the eyes of the subject, the first distance N1 and the second distance N2 may be input by the operator through the operation part 15 described below.

For example, the deflectors 11 may include a plurality of prism lenses having different prism amounts. The deflectors 11 position prism lenses 110 having prism amount corresponding to the prism amount α derived by [Equation 1] from among the plurality of prism lenses in the first optical paths A1. These prism lenses 110 refract the first optical paths A1. Thereby, the deflectors 11 change the first optical paths A1 and form the second optical paths A2. It should be noted that the prism lenses 110 are arranged so as to be base out with respect to the first optical paths A1 as illustrated in FIG. 3.

### (Calculator 12)

The calculator 12 calculates accommodation stimulus amount γ for causing the eyes E to visually recognize the visual target 10 as if it is presented at the second distance N2 based on convergence accommodation amount β of the eyes E caused by the second optical paths A2, the first distance N1 and the second distance N2. To begin with, the convergence accommodation amount β is explained. Accommodation of human eyes is induced from convergence of optic axes. This is a phenomenon called convergence accommodation. Amount of accommodation caused by this phenomenon is referred to as convergence accommodation amount. It is known that 1Δ convergence of the respective human eyes induces accommodation of 0.08D (D: diopter) as a clinical standard value. The calculator 12 calculates the convergence accommodation amount β caused when the eyes E observe a location at the second distance N2 by means of the following equation. It should be noted that when CA/A ratio of the subject has been measured in advance, the calculator 12 may calculate the convergence accommodation amount β by using the CA/A ratio instead of the clinical standard value described above.

$$β = ( \frac{\frac{PD}{2}}{N 2} ) \times 100 \times 0.08 \qquad [ Equation 2 ]$$

β: convergence accommodation amount [D]

The calculator 12 uses the following equation to calculate the accommodation stimulus amount γ based on the convergence accommodation amount β derived from [Equation 2], the first distance N1 and the second distance N2.

$$γ = - ( \frac{1000}{N 2} - \frac{1000}{N 1} - β ) \qquad [ Equation 3 ]$$

γ: accommodation amount [D]

The abovementioned [Equation 3] is explained. If it is assumed that there is no influence of the convergence accommodation amount β, accommodation of 1000/N1 is caused for the eyes E when observing a location at the first distance N1. Similarly, accommodation of 1000/N2 is caused for the eyes E when observing a location at the second distance N2. Thus, it is given that the subjective optometer 1 causes accommodation of 1000/N2−1000/N1 for the eyes E in order to realize a state in which the eyes E observe a location at the second distance N2 by means of the visual target 10 located at the first distance N1.

It should be noted that this accommodation amount is given on the basis of an assumption that there is no influence of the convergence accommodation amount β; therefore, this is excessive accommodation amount in comparison to the state in which the eyes E observe a location at the second distance N2. Thus, the difference between this excessive accommodation amount and the convergence accommodation amount β becomes the accommodation stimulus amount γ.

The calculator 12 may include a storage 120 that previously stores accommodation power information indicating accommodation powers of the eyes, and calculate the convergence accommodation amount of the eyes E caused by the second optical paths A2 based on the accommodation powers indicated by the accommodation power information and the second distance N2. The accommodation power information may be information indicating relationship between human ages and clinical standard values of accommodation powers, for example. As such clinical standard values of accommodation powers, relationship between ages and accommodation powers shown in the following table is known, in general.

TABLE 1

| Ages [years] | Accommodation powers [D] |
|---|---|
| 20 | 8.0 |
| 30 | 7.0 |
| 40 | 4.0 |
| 50 | 1.0 |
| 60 | 0.5 |

The storage 120 stores the relationship between ages of subjects and accommodation powers shown in [Table 1] as the accommodation power information. For example, when the age of a subject is 50 years old (accommodation power 1.0) and the convergence accommodation amount β of the eyes from [Equation 2] is 1.3D, the calculator 12 executes calculation of [Equation 3] with the convergence accommodation amount β=1.0D of the eyes. In other words, the calculator 12 executes calculation using a smaller value between the convergence accommodation amount β from [Equation 2] and the accommodation power indicated in the accommodation power information as the convergence accommodation amount β in [Equation 3]. When the convergence accommodation amount β from [Equation 2] and the accommodation power indicated in the accommodation power information are equal, any of these may be used as the convergence accommodation amount β in [Equation 3]. Further, the age of the subject may be input by the operator through the operation part 15 described above.

The storage 120 may previously store a range in the accommodation powers of the eyes E occupied by the convergence accommodation amount of the eyes E, and the calculator 12 may calculate the convergence accommodation amount β of the eyes E caused by the second optical paths A2 based on an upper limit of the range stored in the storage 120, the accommodation powers indicated by the accommodation power information and the second distance N2. There are cases in which there exists a range in the accommodation powers of the eyes E occupied by the convergence accommodation amount of the eyes E. For example, if the accommodation power of the eyes E is 1.0D and this range is 50%, the convergence accommodation amount of these eyes is 0.5D at most. For example, when the accommodation power indicated in the accommodation power information is 1.0D, this range is 50% and the convergence accommodation amount β from [Equation 2] is 1.3D, the calculator 12 executes calculation of [Equation 3] with the convergence accommodation amount β=0.5D of the eyes. That is, the calculator 12 executes calculation using a smaller value between the convergence accommodation amount β from [Equation 2] and the upper limit of the range in the accommodation powers of the eyes E occupied by the convergence accommodation amount as the convergence accommodation amount β in [Equation 3]. When the convergence accommodation amount β from [Equation 2] and the accommodation power indicated in the accommodation power information are equal, any of these may be used as the convergence accommodation amount β in [Equation 3]. Further, the range may be input by the operator through the operation part 15 described above.

### (Positioning Parts 13)

The positioning parts 13 include spherical lenses 130 and position spherical lenses 130 having spherical powers corresponding to the accommodation stimulus amount γ calculated by the calculator 12 in the second optical paths. The positioning parts 13 may include a plurality of spherical lenses having different spherical powers, for example. The positioning parts 13 position spherical lenses 130 having spherical powers corresponding to the accommodation stimulus amount γ calculated by the calculator 12 from among the plurality of spherical lenses in the second optical paths A2.

### (Controller 14, Operation Part 15 and Display 16)

The controller 14 controls operations of the respective parts. The controller 14 includes a processor and storage, for example. The processor may be CPU (Central Processing Unit), GPU (Graphic Processing Unit) or ASIC (Application Specific Integrated Circuit), for example. The storage includes ROM (Read Only Memory), RAM (Random Access Memory) and HDD (Hard Disc Drive), for example. The storage stores computer programs for executing functions of the respective parts of the subjective optometer 1. The processor executes these computer programs to realize the abovementioned control processing. The operation part 15 is used by the operator for manipulating the subjective optometer 1. The operation part 15 includes various hardware keys (buttons, switches, etc.) provided on a case of the subjective optometer 1 and the like. In a case in which a touch panel display and/or GUI are/is provided, various software keys displayed on them are included in the operation part 15. The display 16 displays information such as spherical powers of the spherical lens 130 and prism amounts of prism lenses 110. The display 16 is configured by a display device such as a liquid crystal display.

### [Operation]

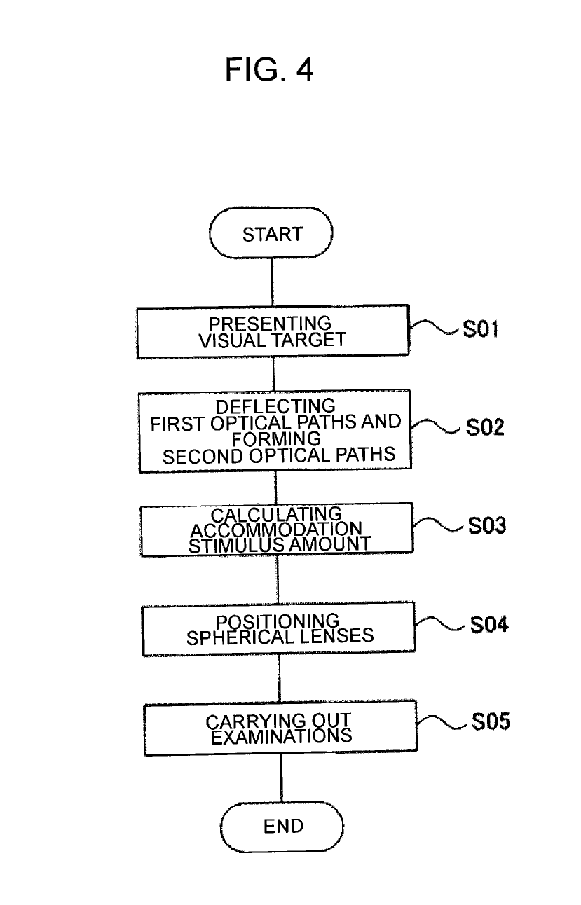

FIG. 4 is a flow chart showing an operation of the subjective optometer 1 of the present embodiment.

### (S01)

The controller 14 controls the visual target 10 to present the visual target 10 at a location the first distance away from the eyes E.

### (S02)

The controller 14 controls the deflectors 11 to deflect the first optical paths A1 formed between the eyes E and the visual target 10, thereby forming the second optical paths A2 for causing the eyes E to observe the visual target 10 as if the visual target 10 is presented at the second distance N2 that is shorter than the first distance N1.

### (S03)

The controller 14 controls the calculator 12 to calculates accommodation stimulus amount γ for causing the eyes E to observe the visual target 10 as if the visual target 10 is presented at the second distance N2 based on convergence accommodation amount β of the eyes E caused by the second optical paths A2, the first distance N1 and the second distance N2.

### (S04)

The controller 14 controls the positioning parts 13 including the spherical lenses to position the spherical lenses 130 having spherical powers corresponding to the accommodation stimulus amount γ calculated by the calculator 12 in the second optical paths A2.

### (S05)

The subjective optometer 1 execute examinations of the eyes in a state in which the spherical lenses 130 are positioned in the second optical paths A2.

### [Actions and Effects]

The actions and effects of the subjective optometer 1 of the present embodiment are explained.

The subjective optometer 1 includes: the visual target 10 that is presented at a location the first distance N1 away from the eyes E; the deflectors 11 that deflect the first optical paths A1 formed between the eyes E and the visual target 10 to form the second optical paths A2 for causing the eyes E to observe the visual target 10 as if the visual target 10 is presented at a location the second distance N2 away, wherein the second distance N2 is shorter than the first distance N1; the calculator 12 that calculates the accommodation stimulus amount γ for causing the eyes E to observe the visual target 10 as if the visual target 10 is presented at the second distance N2 based on the convergence accommodation amount β of the eyes E caused by the second optical paths A2, the first distance N1 and the second distance N2; and the positioning parts 13 that position spherical lenses 130 having spherical powers corresponding to the accommodation stimulus amount γ calculated by the calculator 12 in the second optical paths A2, and the eyes E are examined in a state in which the spherical lenses 130 are positioned in the second optical paths A2. Further, the deflectors 11 may include prism lenses 110 and the prism lenses 110 refract the first optical paths A1 to deflect the first optical paths A1. Further, the calculator 12 includes the storage 120 that previously stores the accommodation power information indicating accommodation powers of the eyes E, and calculates the convergence accommodation amount β of the eyes E caused by the second optical paths A2 based on the accommodation powers indicated by the accommodation power information and the second distance N2. Further, the storage 120 previously stores a range in the accommodation powers of the eyes E occupied by the convergence accommodation amount β of the eyes E, and the calculator 12 calculates the convergence accommodation amount β of the eyes E caused by the second optical paths A2 based on the upper limit of the range stored in the storage 120, the accommodation powers indicated by the accommodation power information and the second distance N2. In this manner, the subjective optometer 1 is capable of causing convergence and convergence accommodation amount β by means of the second optical paths A2 as well as the accommodation stimulus amount β by means of the spherical lenses 130 to the eyes E, and of carrying out examinations in a state in which the eyes E observe the visual target 10 presented at first distance N1 as if the visual target 10 is presented at the second distance N2. Consequently, it is possible to provide subjective optomaters capable of reducing influence of convergence accommodation and carrying out examinations in a state in which convergence and accommodation of eyes are caused in accordance with a predetermined examination distance.

### Second Embodiment

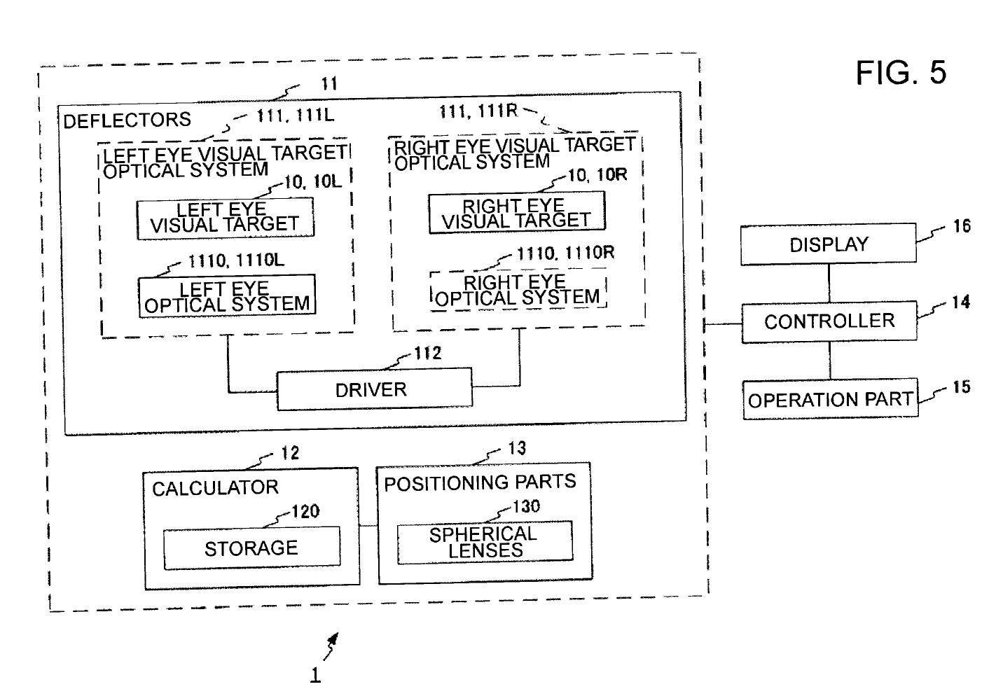

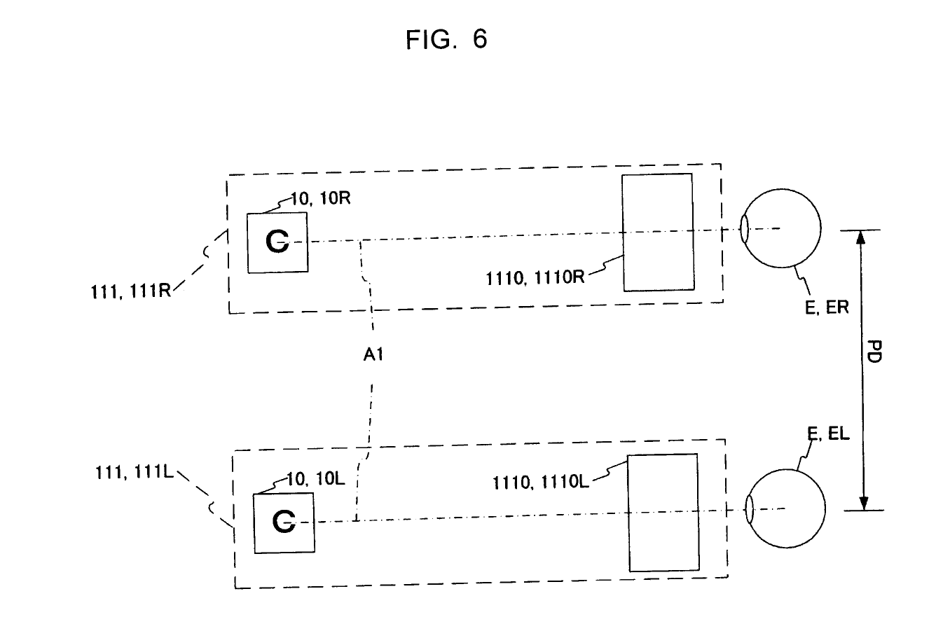

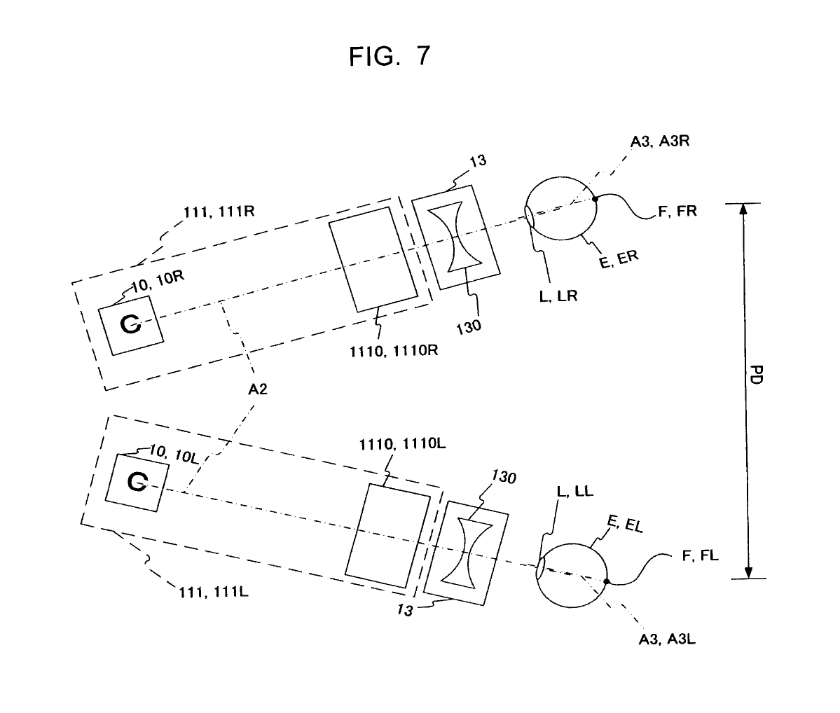

FIG. 5 is a block diagram illustrating a configuration of a subjective optometer 1 according to the second embodiment. FIG. 6 is a schematic diagram illustrating a state in which the eyes E (the left eye EL and the right eye ER) observe the visual target 10 through the first optical paths A1. FIG. 7 is a schematic diagram illustrating a state in which the eyes E observe the visual target 10 through the second optical paths A2. The configurations of the deflectors 11 of the subjective optometer 1 according to the present embodiment are different from those in the first embodiment. Other configurations are similar to the first embodiment.

### [Configuration]

### (Deflectors 11)

The deflectors 11 include: visual target optical systems 111 (left eye visual target optical system 111L and right eye visual target optical system 111R) in which an optical system 1110 (left eye optical system 1110L and right eye optical system 1110R) forming optical distance from the visual target 10 to the eye E and the visual target 10 (left eye visual target 10L and right eye visual target 10R) are integrally configured for each of the left eye and the right eye; and a driver 112 that drives the visual target optical systems 111, and the driver 112 drives the visual target optical systems 111 to deflect the first optical paths A1. The visual target optical systems 111, the visual target 10 and the optical systems 1110 may be configured by applying optical device configuration of a general ophthalmologic apparatus in which left/right-eye-independent type visual targets are installed, for example. The driver 112 may be configured by a general machine mechanism such as motors and gears, for example. Driving amount δ of the visual target optical systems 111 by the driver 112 for each eye is derived from the following equation.

$$δ = {tan}^{- 1} \frac{PD / 2}{N 2} - {tan}^{- 1} \frac{PD / 2}{N 1} \qquad [ Equation 4 ]$$

δ: driving amount [deg]
PD: distance between centers of rotations of eyes of a subject [mm: millimeters]
N1: first distance [mm]
N2: second distance [mm]

From a state in which the optical systems 1110 form optical distance corresponding to the first distance N1 and the visual target 10 is presented to the eyes E, the deflectors 11 receive distance PD between centers of rotations of eyes of a subject, the first distance N1 and the second distance N2 from the controller 14, derives the driving amount δ by means of [Equation 4] and drives the visual target optical systems 111 via the driver 112. The deflectors 11 deflect the first optical paths A1 by this driving to form the second optical paths A2.

As described above, other configurations of the subjective optometer 1 according to the second embodiment are similar to those in the first embodiment. Therefore, as in the first embodiment, the subjective optometer 1 according to the second embodiment calculates accommodation stimulus amount γ for causing the eyes E to visually recognize the visual target 10 as if it is presented at the second distance N2, positions spherical lenses 130 having spherical powers corresponding to the calculated accommodation stimulus amount γ in the second optical paths A2, and carries out examinations of the eyes E in a state in which the spherical lenses 130 are positioned in the second optical paths A2.

### [Actions and Effects]

The actions and effects of the subjective optometer 1 of the present embodiment are explained.

The subjective optometer 1 includes: the visual target 10 that is presented at a location the first distance N1 away from the eyes E; the deflectors 11 that deflect the first optical paths A1 formed between the eyes E and the visual target 10 to form the second optical paths A2 for causing the eyes E to observe the visual target 10 as if the visual target 10 is presented at a location the second distance N2 away, wherein the second distance N2 is shorter than the first distance N1; the calculator 12 that calculates the accommodation stimulus amount γ for causing the eyes E to observe the visual target 10 as if the visual target 10 is presented at the second distance N2 based on the convergence accommodation amount β of the eyes E caused by the second optical paths A2, the first distance N1 and the second distance N2; and the positioning parts 13 that position spherical lenses 130 having spherical powers corresponding to the accommodation stimulus amount γ calculated by the calculator 12 in the second optical paths A2, and the eyes E are examined in a state in which the spherical lenses 130 are positioned in the second optical paths A2. Further, the deflectors 11 may include: the visual target optical systems 111 in which the optical system 1110 forming optical distance from the visual target 10 to the eye E and the visual target 10 are integrally configured for each of the left eye and the right eye; and a driver 112 that drives the visual target optical systems 111, and the driver 112 drives the visual target optical systems 111 to deflect the first optical paths A1. In this manner, the subjective optometer 1 is capable of causing convergence and convergence accommodation amount β by means of the second optical paths A2 as well as the accommodation stimulus amount γ by means of the spherical lenses 130 to the eyes E, and of carrying out examinations in a state in which the eyes E observe the visual target 10 presented at first distance N1 as if the visual target 10 is presented at the second distance N2. Consequently, it is possible to provide subjective optomaters capable of reducing influence of convergence accommodation and carrying out examinations in a state in which convergence and accommodation of eyes are caused in accordance with a predetermined examination distance.

While certain embodiments have been described, these embodiments have been presented by way of example only, and are not intended to limit the scope of the inventions. The embodiments described herein may be embodied in a variety of other forms; furthermore, various omissions, substitutions and changes in the form of the embodiments described herein may be made without departing from the spirit of the invention. The accompanying claims and their equivalents are intended to cover such forms or modifications as would fall within the scope and spirit of the invention.

### EXPLANATION OF SYMBOLS

1 subjective optometer
10 visual target
10L left eye visual target
10R right eye visual target
11 deflectors
12 calculator
13 positioning parts
14 controller
15 operation part
16 display
110 prism lenses
111 visual target optical systems
111L left eye visual target optical system
111R right eye visual target optical system
112 driver
120 storage
130 spherical lenses
1110 optical systems
1110L left eye optical system
1110R right eye optical system
A1 first optical paths
A2 second optical paths
A3 optic axes
E eyes
EL left eye
ER right eye
F central foveas
FL central fovea
FR central fovea
L crystalline lenses
LL crystalline lens
LR crystalline lens
N1 first distance
N2 second distance
P intersection
PD distance between centers of rotations of eyes of a subject
α prism amount
β convergence accommodation amount
γ accommodation stimulus amount
δ driving amount

1. A subjective optometer comprising:
a visual target that is presented at a location a first distance away from eyes;
deflectors that deflect first optical paths formed between the eyes and the visual target to form second optical paths for causing the eyes to observe the visual target as if the visual target is presented at a location a second distance away, wherein the second distance is shorter than the first distance;
a calculator that calculates accommodation stimulus amount for causing the eyes to observe the visual target as if the visual target is presented at the second distance based on convergence accommodation amount of the eyes caused by the second optical paths, the first distance and the second distance; and
positioning parts that comprise spherical lenses and position spherical lenses having spherical powers corresponding to the accommodation stimulus amount calculated by the calculator in the second optical paths,
wherein the eyes are examined in a state in which the spherical lenses are positioned in the second optical paths.

2. The subjective optometer of claim 1, wherein the deflectors comprise prism lenses and the prism lenses refract the first optical paths to deflect the first optical paths.

3. The subjective optometer of claim 1, wherein the deflectors comprise: visual target optical systems in which an optical system forming optical distance from the visual target to an eye and the visual target are integrally configured for each of a left eye and a right eye; and a driver that drives the visual target optical systems, wherein the driver drives the visual target optical systems to deflect the first optical paths.

4. The subjective optometer of claim 1, wherein the calculator comprises a storage that previously stores accommodation power information indicating accommodation powers of the eyes, and calculates the convergence accommodation amount of the eyes caused by the second optical paths based on the accommodation powers indicated by the accommodation power information and the second distance.

5. The subjective optometer of claim 4, wherein the storage previously stores a range in the accommodation powers of the eyes occupied by the convergence accommodation amount of the eyes, and the calculator calculates the convergence accommodation amount of the eyes caused by the second optical paths based on an upper limit of the range stored in the storage, the accommodation powers indicated by the accommodation power information and the second distance.

6. The subjective optometer of claim 2, wherein the calculator comprises a storage that previously stores accommodation power information indicating accommodation powers of the eyes, and calculates the convergence accommodation amount of the eyes caused by the second optical paths based on the accommodation powers indicated by the accommodation power information and the second distance.

7. The subjective optometer of claim 3, wherein the calculator comprises a storage that previously stores accommodation power information indicating accommodation powers of the eyes, and calculates the convergence accommodation amount of the eyes caused by the second optical paths based on the accommodation powers indicated by the accommodation power information and the second distance.

8. The subjective optometer of claim 6, wherein the storage previously stores a range in the accommodation powers of the eyes occupied by the convergence accommodation amount of the eyes, and the calculator calculates the convergence accommodation amount of the eyes caused by the second optical paths based on an upper limit of the range stored in the storage, the accommodation powers indicated by the accommodation power information and the second distance.

9. The subjective optometer of claim 7, wherein the storage previously stores a range in the accommodation powers of the eyes occupied by the convergence accommodation amount of the eyes, and the calculator calculates the convergence accommodation amount of the eyes caused by the second optical paths based on an upper limit of the range stored in the storage, the accommodation powers indicated by the accommodation power information and the second distance.
